(19) United States
(12) Plant Patent Application Publication
(10) Pub. No.: US 2011/0225694 P1
(43) Pub. Date: Sep. 15, 2011

(54) Bermuda grass plant named 'BAR 1CD3'

(50) ;

(75) Inventor: Jacobus De Bruijn, Montech (FR)

(73) Assignee: Barenbrug USA, Inc., Tangent, OR (US)

(21) Appl. No.: 12/661,307

(22) Filed: Mar. 15, 2010

(51) Int. Cl. A01H 5/00

(52) U.S. Cl. PLT/389

(57) ABSTRACT

This invention relates to a new and distinct variety of Bermuda grass plant named ‘BAR 1CD3’, which is primarily characterized by rapid lateral spread that forms a dense sod, minimal thatch, and minimal vertical growth that requires very little mowing, and producing very few seed heads, if any, is disclosed.

### GENUS AND SPECIES

Cynodon dactylon (L.) Persoon

### VARIETY DENOMINATION

‘BAR 1CD3’

### BACKGROUND OF THE NEW PLANT

The present invention relates to a new and distinct Bermuda grass plant designated ‘BAR 1CD3’ and botanically known as Cynodon dactylon (L.) Persoon. This new Bermuda grass was developed from research germplasm collections near Sydney, Australia in 1997. Germplasm selected from the 1997 evaluation was then evaluated in turf plots in southern France from 1998 to 1999. The most promising germplasm was selected and designated ‘BAR 1CD3’. ‘BAR 1CD3’ was then sent to Virginia for further evaluation and selection. A selection was made in 2000 in Virginia for its turf quality. The present invention has been found to be stable and reproduce true to type through successive asexual propagations.

Plant Breeder's Rights for this plant were applied for in The Netherlands in 2001 and granted in 2003. The Netherlands Plant Breeder's Rights application was given the title ‘BARAZUR’. ‘BAR 1CD3’ has not been made publicly available more than one year prior to filing of this application.

### SUMMARY OF THE INVENTION

The following are the most outstanding and distinguishing characteristics of this new cultivar when grown under normal horticultural practices in Raleigh, N.C., Savelletri di Fasano, Italy, Las Cruces, N.Mex., and Wharton, Tex.
1. Rapid lateral spread that forms a dense sod;
2. Minimal thatch;
3. Minimal vertical growth that requires very little mowing; and
4. Very low number of inflorescences (seed heads) produced.

### DESCRIPTION OF THE PHOTOGRAPH AND FIGURE

This new Bermuda grass plant is illustrated by the accompanying photograph taken in Wharton, Tex., on May 6, 2009.

FIG. 1 shows a photo of the invention 30 days after mowing.

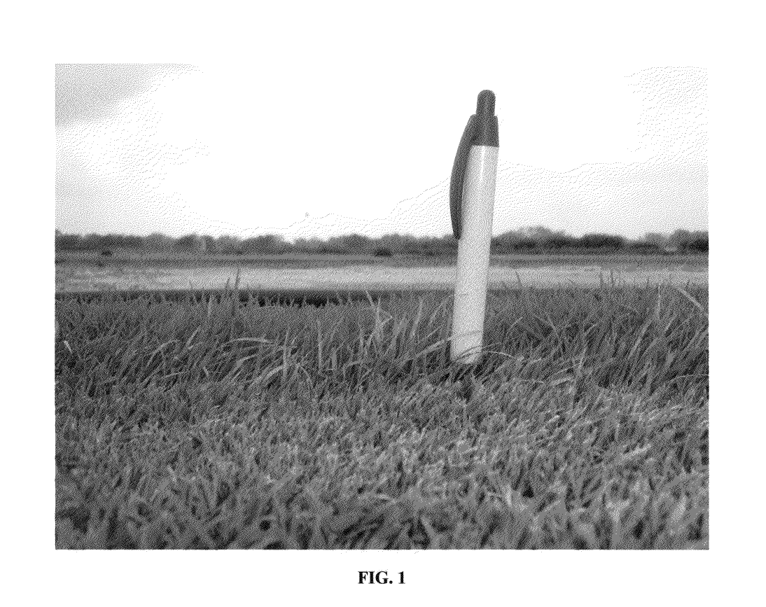

FIG. 2 shows a tree diagram of the genetic linkage distance of ‘BAR 1CD3’ with five other Bermuda grass cultivars.

### DESCRIPTION OF THE NEW VARIETY

The following description of ‘BAR 1CD3’ is based on observations taken during the 2003 to 2009 growing seasons in Raleigh, N.C., Savelletri di Fasano, Italy, Las Cruces, N.Mex., and Wharton, Tex.

### DETAILED BOTANICAL DESCRIPTION

Classification:
   Family.—Poaceae.
   Botanical.—Cynodon dactylon (L.) Persoon.
   Common name.—Bermuda grass.
   Variety name.—‘BAR 1CD3’.
Shoot:
   Density (cm⁻²).—7.3 to 7.4.
Root:
   Rhizome and shoot density (cm cm⁻²).—4.4 to 4.5.
Internode:
   Length (cm).—0.9 to 1.1.
   Diameter (mm).—1.5 to 1.6.
Blade:
   Leaf blade width (mm).—1.6 to 1.8.
Table 1 shows a visual assessment and comparison of ‘BAR 1CD3’ with several commercially available Bermuda grass varieties conducted in Savelletri di Fasano, Italy between 2003 and 2006. The experimental design was a randomized block with four replications. Plot size was 3 m×2 m (0.5 m alleyways between plots). Planting date was Aug. 8, 2003 by sprigging (sprigs in rows) or seeding (1.5 kg 100 m² seed rate). Mowing height of the plot was 30 mm from 2003 to 2005 and 18 mm from 2006 on. The fertilization of the plot included 3.0 kg 100 m⁻² nitrogen per growing season (April to October) and 2.5 kg 100 m⁻² potassium per growing season (April to October). Column one shows the cultivar name, column two shows the propagule type (seeded/vegetative), column three shows the percentage of ground cover during establishment of the grass as of Oct. 11, 2003, column four shows the percent green color and the fall color retention on Dec. 3, 2003, column five shows the spring greenup by percent green color on Mar. 14, 2004, column six shows the spring greenup by percent green color on Apr. 15, 2004, column seven shows the spring greenup by percent green color on May 25, 2004 and row fourteen shows the LSD with an alpha of 0.05.

TABLE 1
Visual Assessment: Percent Ground Cover During Establishment,
Fall Color Retention (2003) and Spring Greenup (2004)

| | | | Fall | | | |
|---|---|---|---|---|---|---|
| | | Percent | Color | Percent Green Color | | |
| | | Ground | Re- | Spring Greenup-2004 | | |
| | | Cover | tention | Mar. | Apr. | May |
| | Propagule | Oct. 11, | Dec. 3, | 14, | 15, | 25, |
| Cultivar | Type | 2003 | 2003 | 2004 | 2004 | 2004 |
| Common | seeded | 100 | 90 | 15 | 61 | 100 |
| Princess 77 | seeded | 99 | 91 | 55 | 89 | 100 |
| Riviera | seeded | 100 | 89 | 56 | 89 | 100 |
| Yukon | seeded | 88 | 39 | 20 | 91 | 100 |
| Tifway 419 | vegetative | 69 | 91 | 70 | 95 | 100 |
| Tifdwarf | vegetative | 30 | 90 | 10 | 38 | 84 |
| Santa Ana | vegetative | 71 | 92 | 65 | 95 | 100 |
| Tiftsport | vegetative | 33 | 91 | 60 | 83 | 99 |
| ‘BAR 1CD3’ | vegetative | 40 | 68 | 50 | 66 | 95 |
| LSD (P = 0.05) | | 21 | 6 | 6 | 19 | 12 |

Table 2 below shows a visual assessment and comparison of ‘BAR 1CD3’ with several commercially available Bermuda grass varieties. The comparison was made in the spring of 2006 in Savelletri di Fasano, Italy between 2003 and 2006. The experimental design was a randomized block with four replications. Plot size was 3 m×2 m (0.5 m alleyways between plots). Planting date was Aug. 8, 2003 by sprigging (sprigs in rows) or seeding (1.5 kg 100 m² seed rate). Mowing height of the plot was 30 mm from 2003 to 2005 and 18 mm from 2006 on. The fertilization of the plot included 3.0 kg 100 m⁻² nitrogen per growing season (April to October) and 2.5 kg 100 m⁻² potassium per growing season (April to October). Column one shows the cultivar name, column two shows the percent green color on Mar. 2, 2006, column three shows the percent green color on Mar. 15, 2006, column four shows the percent green color on Mar. 30, 2006, and row thirteen shows the LSD with an alpha of 0.05

TABLE 2
Spring Greenup 2006

| | Percent Green Color | | |
|---|---|---|---|
| Cultivar | Mar. 2, 2006 | Mar. 15, 2006 | Mar. 30, 2006 |
| Common | 0 | 5 | 61 |
| Princess | 0 | 0 | 60 |
| Riviera | 0 | 10 | 79 |
| Yukon | 0 | 6 | 75 |
| Tifway 419 | 1 | 8 | 84 |
| Tifdwarf | 0 | 0 | 63 |
| Santa Ana | 0 | 9 | 78 |
| Tifsport | 0 | 0 | 74 |
| ‘BAR 1CD3’ | 0 | 0 | 74 |
| LSD (P = 0.05) | 2 | 4 | 10 |

Table 3 below shows a visual assessment and comparison of ‘BAR 1CD3’ with several commercially available Bermuda grass varieties. The comparison was made in the fall of 2004, 2005, and 2006 in Savelletri di Fasano, Italy between 2003 and 2006. The experimental design was a randomized block with four replications. Plot size was 3 m×2 m (0.5 m alleyways between plots). Planting date was Aug. 8, 2003 by sprigging (sprigs in rows) or seeding (1.5 kg 100 m² seed rate). Mowing height of the plot was 30 mm from 2003 to 2005 and 18 mm from 2006 on. The fertilization of the plot included 3.0 kg 100 m⁻² nitrogen per growing season (April to October) and 2.5 kg 100 m⁻² potassium per growing season (April to October). Column one shows the cultivar name, column two shows the turf quality (9=best and 1=poorest) on Sep. 23, 2004, column three shows the turf quality (9=best and 1=poorest) on Sep. 29, 2005, column four shows the turf color on Sep. 23, 2004 (9=dark green and 1=light green), and row thirteen shows the LSD with an alpha of 0.05.

TABLE 3
Visual Assessment: Turf Quality and Turf Color

| | | | Color |
|---|---|---|---|
| | Turf Quality | | (9 = dark green, |
| | (9 = best, 1 = poorest) | | 1 = light green) |
| Cultivar | Sep. 23, 2004 | Sep. 29, 2005 | Sep. 23, 2004 |
| Common | 5.4 | 4.0 | 5.3 |
| Princess 77 | 7.1 | 6.0 | 7.0 |
| Riviera | 6.8 | 4.8 | 6.8 |
| Yukon | 7.3 | 5.5 | 6.8 |
| Tifway 419 | 7.8 | 5.8 | 7.6 |
| Tifdwarf | 7.8 | 7.0 | 7.8 |
| Santa Ana | 7.5 | 6.0 | 7.5 |
| Tifsport | 7.8 | 6.1 | 8.0 |
| ‘BAR 1CD3’ | 8.3 | 7.3 | 8.4 |
| LSD (P = 0.05) | 0.4 | 0.5 | 0.4 |

Table 4 below shows a comparison of morphological characteristics recorded in 2004. The comparison was made in Savelletri di Fasano, Italy between 2003 and 2006. The experimental design was a randomized block with four replications. Plot size was 3 m×2 m (0.5 m alleyways between plots). Planting date was Aug. 8, 2003 by sprigging (sprigs in rows) or seeding (1.5 kg 100 m² seed rate). Mowing height of the plot was 30 mm from 2003 to 2005 and 18 mm from 2006 on. The fertilization of the plot included 3.0 kg 100 m⁻² nitrogen per growing season (April to October) and 2.5 kg 100 m⁻² potassium per growing season (April to October). Column one shows the cultivar name, column two shows the shoot density (cm⁻²) (one 70 mm core sample per plot), column three shows the rhizome and stolon density (cm cm⁻²) (one 70 mm core sample per plot), column four shows the internode length (cm) (20 internodes were measured per plot), column five shows the internode diameter (mm) (20 internodes were measured per plot), column six shows the leaf blade width (mm) (20 fully expanded leaves were measured per plot), and row twelve shows the LSD with an alpha of 0.05.

TABLE 4
Comparative Turfgrass Morphological Characteristics
Recorded in 2004

| | | Rhizome | | | |
|---|---|---|---|---|---|
| | Shoot | and Stolon | Internode | Internode | Leaf Blade |
| | Density | Density | Length | Diameter | Width |
| Cultivar | (cm⁻²) | (cm cm⁻²) | (cm) | (mm) | (mm) |
| Common | 3.6 | 0.4 | 1.4 | 1.3 | 1.5 |
| Princess 77 | 6.6 | 0.8 | 1.2 | 0.9 | 1.4 |
| Riviera | 6.7 | 0.9 | 1.4 | 1.3 | 1.3 |
| Yukon | 5.1 | 1.0 | 1.3 | 1.3 | 1.4 |
| Tifway 419 | 5.9 | 1.1 | 1.5 | 1.5 | 1.3 |
| Tifdwarf | 9.4 | 2.2 | 1.2 | 1.3 | 1.2 |
| Santa Ana | 8.2 | 3.0 | 1.5 | 1.4 | 1.1 |
| Tifsport | 11.2 | 2.7 | 1.8 | 1.4 | 1.2 |
| ‘BAR 1CD3’ | 7.3 | 4.5 | 1.1 | 1.6 | 1.8 |
| LSD (P = 0.05) | 2.7 | 1.8 | 0.3 | 0.4 | 0.2 |

Table 5 below shows a comparison of morphological characteristics recorded in 2005. The comparison was made in Savelletri di Fasano, Italy between 2003 and 2006. The experimental design was a randomized block with four replications. Plot size was 3 m×2 m (0.5 m alleyways between plots). Planting date was Aug. 8, 2003 by sprigging (sprigs in rows) or seeding (1.5 kg 100 m² seed rate). Mowing height of the plot was 30 mm from 2003 to 2005 and 18 mm from 2006 on. The fertilization of the plot included 3.0 kg 100 m⁻² nitrogen per growing season (April to October) and 2.5 kg 100 m⁻² potassium per growing season (April to October). Column one shows the cultivar name, column two shows the shoot density (cm⁻²) (one 70 mm core sample per plot), column three shows the rhizome and stolon density (cm cm ⁻²) (one 70 mm core sample per plot), column four shows the internode length (cm) (20 internodes were measured per plot), column five shows the internode diameter (mm) (20 internodes were measured per plot), column six shows the leaf blade width (mm) (20 fully expanded leaves were measured per plot), and row twelve shows the LSD with an alpha of 0.05.

TABLE 5
Comparative Turfgrass Morphological Characteristics
Recorded In 2005

| | | Rhizome | | | |
|---|---|---|---|---|---|
| | Shoot | and Stolon | Internode | Internode | Leaf Blade |
| | Density | Density | Length | Diameter | Width |
| Cultivar | (cm⁻²) | (cm cm⁻²) | (cm) | (mm) | (mm) |
| Common | 3.7 | 0.2 | 1.3 | 1.7 | 1.8 |
| Princess 77 | 9.7 | 0.7 | 1.2 | 0.7 | 1.4 |
| Riviera | 5.4 | 0.4 | 1.0 | 1.1 | 1.4 |
| Yukon | 6.0 | 0.9 | 1.7 | 1.5 | 1.7 |
| Tifway 419 | 5.6 | 1.6 | 1.2 | 1.6 | 1.4 |
| Tifdwarf | 10.5 | 2.7 | 1.1 | 1.1 | 1.2 |
| Santa Ana | 13.1 | 2.9 | 1.2 | 1.5 | 1.0 |
| ‘BAR 1CD3’ | 7.4 | 4.4 | 0.9 | 1.5 | 1.6 |
| Tifsport | 12.8 | 2.8 | 1.4 | 1.4 | 1.1 |
| LSD (P = 0.05) | 3.8 | 1.1 | 0.8 | 0.4 | 0.1 |

Table 6 shows the average plant heights (cm) after 56 days and 77 days without mowing the plots on May 6, 2009 and May 27, 2009. The date of planting was Jun. 13, 2008 in Wharton, Tex. Column one shows the cultivar name, column two shows the plant height after 56 days (cm), column three shows the plant height after 77 days (cm), and row ten shows the LSD with an alpha of 0.05. The statistical analysis is based on eight observations per plot. All varieties in Table 6 are vegetatively propagated.

TABLE 6
Average Plant Height (cm) After 56 Days And 77 Days
Without Mowing The Plots. Planting Date Jun. 13, 2008

| | 56 days | 77 days |
|---|---|---|
| | May 6, 2009 | May 27, 2009 |
| Cultivar | (cm) | (cm) |
| ‘BAR 1CD3’ | 6.91 | 14.61 |
| Tifton 419 | 10.72 | 17.70 |
| Plateau | 12.86 | 17.86 |
| Patriot | 14.84 | 23.81 |
| Celebration | 16.59 | 31.91 |
| LSD (P = 0.05) | 1.05 | 1.39 |

Table 7 shows a comparison of the genetic color, leaf texture, and number of seed heads. Data was collected from plots planted in the summer of 2007 in Raleigh, N.C. Column one shows the cultivar name, column two shows the propagule type, column three shows the genetic color on Sep. 19, 2007 with (9=dark green and 1=light green), column four shows the genetic color on Oct. 5, 2007 with (9=dark green and 1=light green), column five shows the genetic color in September, 2008 with (9=dark green and 1=light green), column six shows the leaf texture on Oct. 5, 2007 with a score of nine (9) equaling the finest texture, column seven shows the leaf texture in September, 2008 with a score of nine (9) equaling the finest texture, column eight shows the number of seed heads on Oct. 7, 2007 with a score of nine (9) equaling no seed heads produced and one (1) equaling the highest number of seed heads produced, column nine shows the number of seed heads on Jul. 21, 2008 with a score of nine (9) equaling no seed heads produced and one (1) equaling the highest number of seed heads produced, column ten shows the number of seed heads in Oct. 26, 2008 with a score of nine (9) equaling no seed heads were produced and one (1) equaling the highest number of seed heads produced, and column eleven shows the average number of seeds heads for 2007 and 2008, with a score of nine (9) equaling no seed heads were produced and one (1) equaling the highest number of seed heads produced. Rows 14 through 16 show the statistical analyses of the data in Table 7, including the LSD with an alpha of 0.05, the standard deviation, and Coefficient of Variation (CV).

TABLE 7
Shows a Comparison of the Average Genetic Color, Leaf Texture
and Number of Seed Heads in 2007 and 2008

| | | | | | Leaf Texture | |
|---|---|---|---|---|---|---|
| | | Genetic Color | | | (9 = finest | |
| | | (9 = darkest green) | | | texture) | |
| | | | | Sep- | | Sep- |
| | | | | tem- | | tem- |
| | Propagule | Sep. 19, | Oct. 5, | ber of | Oct. 5, | ber of |
| Entry | Type | 2007 | 2007 | 2008 | 2007 | 2008 |
| ‘BAR 1CD3’ | vegetative | 8.3 | 8.0 | 8.0 | 9.0 | 9.0 |
| Riviera | seeded | 6.0 | 5.7 | 6.3 | 7.0 | 6.7 |
| Princess 77 | seeded | 7.0 | 6.3 | 7.0 | 7.7 | 7.3 |
| Midlawn | vegetative | 7.0 | 6.3 | 6.0 | 7.3 | 9.0 |
| Tifway | vegetative | 8.0 | 7.3 | 8.0 | 8.3 | 9.0 |
| Premier | vegetative | 8.3 | 7.0 | 7.7 | 9.0 | 8.7 |
| Sunsport | seeded | 6.3 | 5.0 | 5.3 | 6.7 | 5.3 |
| Patriot | vegetative | 9.0 | 8.0 | 8.3 | 8.0 | 8.3 |
| Yukon | seeded | 6.7 | 6.3 | 7.3 | 6.7 | 6.7 |
| Tifton 11 | vegetative | 8.3 | 7.3 | 7.3 | 7.7 | 7.3 |

| | | | | | |
|---|---|---|---|---|---|
| LSD (P = 0.05) | 0.8 | 1.2 | 0.9 | 0.8 | 1.1 |
| Standard Deviation | 0.5 | 0.7 | 0.5 | 0.5 | 0.7 |
| CV | 7.13 | 11.28 | 8.1 | 6.73 | 10.1 |

| | | | | | Average |
|---|---|---|---|---|---|
| | | | | | Number |
| | | | | | of |
| | | Number of Seed Heads | | | Seed |
| | | (9 = no seed heads) | | | Heads |
| | Propagule | Oct. 7, | Jul. 21, | Oct. 26, | 2007- |
| Entry | Type | 2007 | 2008 | 2008 | 2008 |
| ‘BAR 1CD3’ | vegetative | 8.7 | 9.0 | 9.0 | 8.9 |
| Riviera | seeded | 7.7 | 6.7 | 7.7 | 7.3 |
| Princess 77 | seeded | 5.7 | 4.0 | 6.0 | 5.2 |
| Midlawn | vegetative | 8.3 | 7.3 | 9.0 | 8.2 |
| Tifway | vegetative | 8.3 | 7.0 | 9.0 | 8.1 |
| Premier | vegetative | 8.0 | 8.7 | 8.7 | 8.4 |
| Sunsport | seeded | 4.0 | 3.3 | 5.7 | 4.3 |
| Patriot | vegetative | 7.3 | 9.0 | 6.7 | 7.7 |
| Yukon | seeded | 7.7 | 5.7 | 8.0 | 7.1 |
| Tifton 11 | vegetative | 8.7 | 8.3 | 9.0 | 8.7 |

| | | | | |
|---|---|---|---|---|
| LSD (P = 0.05) | 1.1 | 1.1 | 1.5 | 0.8 |
| Standard Deviation | 0.7 | 0.7 | 0.9 | 0.5 |
| CV | 10.47 | 12 | 13.17 | 7.44 |

Table 8 shows a non-replicated comparison of turf quality and genetic color. Data was collected in the summer and fall of 2008 from plots planted on Jun. 13, 2008 in Wharton, Tex. Column one shows the cultivar name, column two shows the turf quality (9=best and 1=poorest) on Jul. 11, 2008, column three shows the turf quality (9=best and 1=poorest) on Jul. 22, 2008, column four shows the turf quality (9=best and 1=poorest) on Aug. 29, 2008, column five column the turf quality (9=best and 1=poorest) on Sep. 19, 2008, column six shows the turf quality (9=best and 1=poorest) on Oct. 2, 2008, column seven shows the average turf quality, column eight shows the genetic color on Jul. 11, 2008 (9=dark green and 1=light green), column nine shows the genetic color on Jul. 22, 2008 (9=dark green and 1=light green), column ten shows the genetic color on Aug. 29, 2008 (9=dark green and 1=light green), column eleven shows the genetic color on Sep. 19, 2008 (9=dark green and 1=light green), column twelve shows the genetic color on Oct. 2, 2008 (9=dark green and 1=light green), and column thirteen shows the average genetic color.

TABLE 8
Comparison of Turf Quality and Genetic Color
Data is from Wharton, Texas. Planting date: Jun. 13, 2008

| | Turf Quality | | | | | |
|---|---|---|---|---|---|---|
| | (9 = best turf quality) | | | | | |
| | Jul. 11, | Jul. 22, | Aug. 29, | Sep. 19, | Oct. 2, | |
| Cultivar | 2008 | 2008 | 2008 | 2008 | 2008 | Average |
| Plateau | 7 | 7 | 7 | 7 | 7 | 7.0 |
| Patriot | 7 | 7 | 5 | 6 | 5 | 6.0 |
| ‘BAR 1CD3’ | 7 | 7 | 8 | 7 | 6 | 7.0 |
| Celebration | 7 | 7 | 6 | 6 | 6 | 6.4 |
| Tifton 419 | 7 | 7 | 7 | 6 | 6 | 6.6 |
| | Genetic Color | | | | | |
| | (9 = dark green) | | | | | |
| | Jul. 11, | Jul. 22, | Aug. 29, | Sep. 19, | Oct. 2, | |
| Cultivar | 2008 | 2008 | 2008 | 2008 | 2008 | Average |
| Plateau | 7 | 8 | 8 | 8 | 7.6 | 7.7 |
| Patriot | 6 | 6 | 7 | 6 | 6.2 | 6.2 |
| ‘BAR 1CD3’ | 7 | 8 | 8 | 7 | 7.4 | 7.5 |
| Celebration | 7 | 7 | 7 | 8 | 7.1 | 7.2 |
| Tifton 419 | 6 | 6 | 6 | 6 | 6.1 | 6.0 |

Table 9 shows a comparison of the number of seed heads produced and percent ground cover. Data was collected in the summer and fall of 2008 from plots planted on Jun. 13, 2008 in Wharton, Tex. Column one shows the cultivar name, column two shows the seed head number on Jul. 11, 2008 with a score of nine (9) equaling the highest number of seed heads produced, column three shows the seed head number on Jul. 22, 2008 with a score of nine (9) equaling the highest number of seed heads produced, column four shows the seed head number on Aug. 29, 2008 with a score of nine (9) equaling the highest number of seed heads produced, column five shows the seed head number on Sep. 19, 2008 with a score of nine (9) equaling the highest number of seed heads produced, column six shows the seed head number on Oct. 2, 2008 with a score of nine (9) equaling the highest number of seed heads produced, column seven shows the average number of seed head produced, column eight shows the percent ground cover on Jul. 11, 2008, column nine shows the percent ground cover on Jul. 22, 2008, column ten shows the percent ground cover on Aug. 29, 2008, column eleven shows the percent ground cover on Sep. 19, 2008, and column twelve shows the percent ground cover on Oct. 2, 2008.

TABLE 9
Comparison of Seed Head Production and Percent Ground Cover
Data is from Wharton, Texas. Planting date: Jun. 13, 2008

| | Seed Head Number | | | | | |
|---|---|---|---|---|---|---|
| | (9 = highest number of seed heads produced) | | | | | |
| | Jul. 11, | Jul. 22, | Aug. 29, | Sep. 19, | Oct. 2, | |
| Cultivar | 2008 | 2008 | 2008 | 2008 | 2008 | Average |
| Plateau | 0 | 0 | 3 | 3 | 3 | 1.8 |
| Patriot | 0 | 0 | 0 | 0 | 9 | 1.8 |
| ‘BAR 1CD3’ | 0 | 0 | 0 | 0 | 0 | 0.0 |
| Celebration | 0 | 0 | 0 | 0 | 0 | 0.0 |
| Tifton 419 | 0 | 0 | 0 | 0 | 0 | 0.0 |

Percent Ground Cover

| | Jul. 11, | Jul. 22, | Aug. 29, | Sep. 19, | Oct. 2, |
|---|---|---|---|---|---|
| Cultivar | 2008 | 2008 | 2008 | 2008 | 2008 |
| Plateau | 35 | 75 | 100 | 100 | 100 |
| Patriot | 90 | 100 | 100 | 100 | 100 |
| ‘BAR 1CD3’ | 65 | 90 | 100 | 100 | 100 |
| Celebration | 70 | 95 | 100 | 100 | 100 |
| Tifton 419 | 50 | 85 | 100 | 100 | 100 |

Table 10 shows a written comparison of various Bermuda grass varieties when compared with ‘BAR 1CD3’. Data is from Wharton, Tex., with a planting date of Jun. 13, 2008. As shown in Table 10, ‘BAR 1CD3’ is distinguished from other Bermuda grass varieties by its blue green color, very low vertical growth, tight and dense growth, short leaf, little or no scalping when mowed, and no seed head production.

TABLE 10
Comparison of Characteristics of Various Bermuda Grass Varieties

| | |
|---|---|
| Plateau | Blue green color; low vertical growth; tight and dense; |
| | short leaf; little or no scalping when mowed; some |
| | seed heads produced. |
| Patriot | Yellow green color; tall vertical growth; thin in turf; |
| | course leaf; scalped when mowed; numerous |
| | seed heads produced. |
| ‘BAR 1CD3’ | Blue green color; very low vertical growth; tight and |
| | dense; short leaf; little or no scalping when mowed; |
| | no seed heads produced. |
| Celebration | Blue green color; medium vertical growth; thin; coarse leaf. |
| Tifton 419 | Light green in color; tall growth; thin, fine leaf blades, |
| | scalping when mowed. |

An analysis of the genetic relationship between ‘BAR 1CD3’ and various Bermuda grass samples was conducted in the fall of 2009. Amplified Fragment Length Polymorphism (AFLP) markers were used in the analysis. From each DNA-sample, three independent EcoRI/MseI pre-amplifications were prepared. This rendered a total of eighteen pre-amplifications for analysis and allowed a check of the reproducibility of the AFLP-protocol when the same DNA-sample was used repeatedly for analysis. For each pre-amplification, eight AFLP primer combinations were used. Six primer combinations were used as taught by Wu et al., Genome 47:689-696 (2004) and are shown in Table 11. Primer combination e-GCTG/m-CAG, as taught by Wu et al. (2004) was replaced by the primer combination e-ACA/m-CAG to avoid the necessity to carry out two preamplifications for each sample in triplicate. Primer combination e-AGT/m-CAG was replaced by primer combination e-ACA/m-CAT, to avoid unnecessary delay in the completion of the analysis. An ABI Prism 3130x1 capillary sequencer was used for the AFLP-fragment separation and detection. GeneScan 500 Rox labelled size standard was loaded in each run to allow fragment sizing. The fluorescent AFLP fingerprints were scored using Genemapper v. 3.7 (Applied Biosystems) and the presence or absence of each marker in each sample was recorded. As shown in Table 12, the presence or absence of each marker was used to construct a scoring table where a score of 1 equalled presence and a score of 0 equalled absence for data analysis. Genetic similarity between pairs of samples was estimated using the percentage of shared AFLP bands. This coefficient varies between 0 (complete dissimilarity) and 1 (complete similarity). Calculations were carried out in STATISTICA v8.0 (Stat Soft Inc., Tulsa, Okla., USA).

TABLE 11
Restriction Enzymes and Selective Nucleotides Used,
Primer Combinations as cited by Wu et al. (2004)
EcoRI MseI

PC1* ACT CAG
PC2* AAC CAG
PC3 ACA CAG
PC4* ACT CAT
PC5* AAC CAT
PC6* ACT CAC
PC7* AAC CAC
PC8 ACA CAT

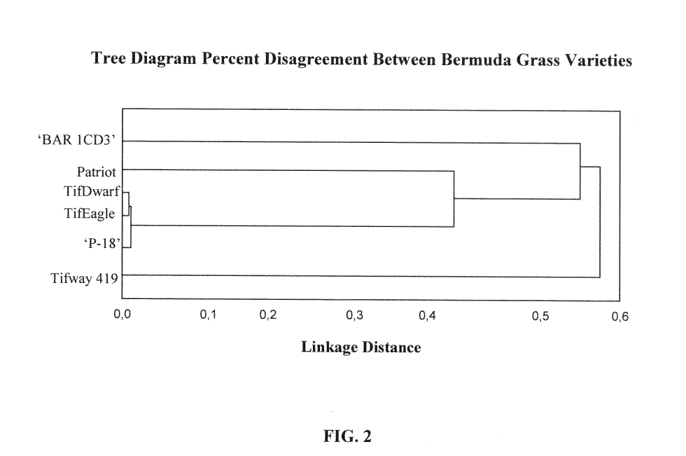

As shown in Table 11, eight primer combinations were used. These eight primer combinations rendered 373 polymorphic AFLP-bands for analysis. The fingerprints generated for replicated samples were compared. In most cases three repetitions were available for analysis for a given primer combination. Only a few differences were encountered when replicated samples were compared: for Patriot only one AFLP-band (PC8_a17) was not consistent among the three replications. For ‘BAR 1CD3’ only the AFLP-band PC3_a135 was not consistent among the three replications and for Tifway 419 only the AFLP-band PC7_a135 was not consistent. For Tifdwarf, Celebration, TifEagle and ‘P-18’ the replicates were identical. This demonstrated a high reliability of the results obtained.
As shown in Table 12, the analysis shows that the Bermuda grass varieties Tifdwarf, TifEagle and ‘P-18’ display an extremely high degree of similarity. No single polymorphism was detected between samples Tifdwarf and TifEagle among the 373 AFLP-bands screened. One single polymorphism (for band PC6_a129) was found between Tifdwarf and TifEagle, on the one side, and ‘P-18’ on the other side.
The genetic relationships among the Bermuda grass samples tested have also been represented using Unweighted Pair Group Method with Arithmetic Mean clustering UPGMA (FIG. 2). As shown in FIG. 2, four clusters are evident with Tifway 419 being the most dissimilar, and TifDwarf, TifEagle, and ‘P-18’ being the most similar of the group.

TABLE 12
Analysis of the Genetic Similarity of Bermuda Grass Varieties

| | | | | | | Tifway |
|---|---|---|---|---|---|---|
| | ‘BAR 1CD3’ | Patriot | TifDwarf | TifEagle | ‘P-18’ | 419 |
| ‘BAR 1CD3’ | 0.000 | 0.485 | 0.606 | 0.610 | 0.614 | 0.608 |
| Patriot | 0.485 | 0.000 | 0.408 | 0.412 | 0.416 | 0.535 |
| TifDwarf | 0.606 | 0.408 | 0.000 | 0.004 | 0.008 | 0.577 |
| TifEagle | 0.610 | 0.412 | 0.004 | 0.000 | 0.004 | 0.581 |
| ‘P-18’ | 0.614 | 0.416 | 0.008 | 0.004 | 0.000 | 0.581 |
| Tifway 419 | 0.608 | 0.535 | 0.577 | 0.581 | 0.581 | 0.000 |

What is claimed is:

1. A new and distinct Bermuda grass plant as described and illustrated herein.
